(19) United States
(12) Patent Application Publication
(10) Pub. No.: US 2025/0235213 A1
(43) Pub. Date: Jul. 24, 2025

(54) ANASTOMOSIS CONNECTOR, A GROUP OF PARTS AND AN APPLICATOR

(71) Applicant: UMC UTRECHT HOLDING B.V., Utrecht (NL)

(72) Inventors: Wilhelmus Josephus Leonardus Suyker, Utrecht (NL); Paulus Thomas Wilhelmus Suyker, Amsterdam (NL); Harmen Matthijs Wassink, Amersfoort (NL); Jeroen Van Den Berg, Putten (NL); Froukje Ellen Euwe, Utrecht (NL); Jesse Mattan Bosma, Utrecht (NL); Leonard Jan Van Schelven, Houten (NL); Paul Frederik Gründeman, Grou (NL); Monica Gianoli, Zeist (NL)

(21) Appl. No.: 18/703,412

(22) PCT Filed: Oct. 24, 2022
(86) PCT No.: PCT/NL2022/050599

§ 371 Date: Apr. 22, 2024

(30) Foreign Application Priority Data
Oct. 25, 2021 (NL) 2029502

(51) Int. Cl. A61B 17/115; A61B 17/00; A61B 17/11

(52) U.S. Cl. CPC A61B 17/115; A61B 2017/00477; A61B 2017/00862; A61B 2017/1107; A61B 2017/1139; A61B 2017/1157

(57) ABSTRACT

An anastomosis connector for being mounted to a flexible vessel, such as a blood vessel, comprises a non-expandable annular body having an inner wall surrounding an anastomotic orifice, an outer wall surrounding the inner wall, a front side and a back side which extend between the inner wall and the outer wall, and a plurality of staple pins configured to be bent permanently from a starting position to a final position and located at a distance from each other in circumferential direction of the annular body. The staple pins project from the annular body and point in a direction away from the back side. The distance between the front side and the back side at the inner wall of the annular body is smaller than the distance between the inner wall and the outer wall.

### CROSS-REFERENCE TO RELATED APPLICATIONS

This Application is a Section 371 National Stage Application of International Application No. PCT/NL2022/050599, filed Oct. 24, 2022 and published as WO 2023/075596 A1 on May 4, 2023, in English.

### BACKGROUND

The present invention relates an anastomosis connector for being mounted to a flexible vessel, such as a blood vessel, comprising a non-expandable annular body having an inner wall surrounding an anastomotic orifice, an outer wall surrounding the inner wall, a front side and a back side which extend between the inner wall and the outer wall, a plurality of staple pins configured to be bent permanently from a starting position to a final position and located at a distance from each other in circumferential direction of the annular body, wherein the staple pins project from the annular body and point in a direction away from the back side.

### SUMMARY

An improved anastomosis connector is characterized in that the distance between the front side and the back side at the inner wall of the annular body is smaller than the distance between the inner wall and the outer wall.

An advantage of the anastomosis connector is that it is suitable for extraluminal placement on a vessel by fixing the staple pins around an opening in a vessel wall such that the annular body remains outside the vessel and its front side is directed away from the opening. This provides the opportunity to mount two of such anastomosis connectors to respective vessels which should be connected to each other and subsequently fix the anastomosis connectors to each other such that their front sides face each other. The relatively large distance between the inner wall and the outer wall of the annular bodies can provide sufficient material to fix the annular bodies to each other, possibly by means of a separate fixing member. Besides, the relatively large distance between the inner wall and the outer wall of the annular body keeps the annular body at the outer side of a vessel.

The vessel to which the anastomosis connector can be mounted may be a vessel graft, either a prosthetic or natural vessel and a native vessel. For example, the anastomosis connector can be positioned on a blood vessel at an opening thereof, e.g. an existing open end of a vessel or a created opening in a vessel wall, wherein the staple pins are initially directed towards the vessel, after which the staple pins are bent such that they penetrate into an edge portion of the vessel which surrounds the opening.

The relatively small distance between the front side and the back side at the inner wall of the annular body ensures a relatively small exposed surface of the annular body at the inner side of a vessel to which it is mounted, hence minimizing contact between a liquid in the vessel and material of the anastomosis connector. Additionally, it appears that when two anastomosis connectors are mounted to respective vessels and subsequently fixed to each other, tissues of these vessels which surround the corresponding anastomotic orifices can grow to each other over the respective inner walls and unify within a relatively short period. This effect increases with decreasing distances between the front side and the back side at the inner wall of the annular bodies.

A further advantage of the anastomosis connector is that a surgeon who constructs the anastomosis maintains visual control during each step of constructing the anastomosis.

Particularly, in case of a coronary anastomosis the mentioned advantages are of great relevance, since the anastomosis connectors can be mounted to respective blood vessels and fixed to each other through endoscopic surgery, whereas small exposed surfaces of the annular bodies at the inner side of the blood vessels minimize the risk of a thrombus. Coronary revascularization through endoscopic surgery will dramatically reduce the number of complications and recovery time compared to open chest surgery. Nevertheless, it is also possible to use the invention to create anastomoses between other flexible vessels or flexible hollow structures.

The anastomosis connector may be made from a biocompatible metal, such as stainless steel 316LVM or the like.

The distance between the front side and the back side at the inner wall of the annular body may be smaller than 50%, preferably smaller than 25%, or even smaller than 10%, of the distance between the inner wall and the outer wall. For example, the distance between the front side and the back side at the inner wall of the annular body may be smaller than 0.25 mm and preferably smaller than 0.15 mm. The distance may be 0.10 mm, for example. Furthermore, the length of each of the staple pins in the starting position thereof may be shorter than the distance between the inner wall and the outer wall.

It is noted that the distance between the front side and the back side of the annular body is not necessarily only at the inner wall smaller than the distance between the inner wall and the outer wall, but also the distance between the front side and the back side remote from the inner wall may be smaller than the distance between the inner wall and the outer wall. The length of each of the staple pins may be larger than its width as measured in circumferential direction about the anastomotic orifice or larger than twice, three times, four times or five times.

The distance between the inner wall and the outer wall may be larger than the thickness of any of the staple pins as measured in a direction from the inner wall to the outer wall at the location of the corresponding staple pin. This provides the opportunity to clamp the vessel wall to which the anastomosis connector is mounted against the annular body by means of the curved staple pins.

In a preferred embodiment the staple pins are configured to be bent in outward direction of the annular body, since this allows the staple pins to enter an opening in a vessel where the anastomosis connector should be placed still without penetrating the vessel wall and to penetrate the vessel wall only after being partly bent and free ends of the staple pins move back towards the annular body. The vessel wall is then located between the free ends of the staple pins and the annular body which facilitates penetration. Without the presence of the annular body the vessel wall might be pushed away without penetrating the vessel wall.

The staple pins may be located at the inner wall of the annular body so as to minimize the exposed surface of the annular body to the interior of a vessel to which it is to be mounted.

The inner wall between each pair of neighbouring staple pins in circumferential direction of the annular body may extend substantially linearly. This means that in a mounted condition on a vessel the edge around the opening of a vessel which usually extends linearly between two staple pins can follow the inner wall accurately, hence minimizing the risk of leakage. In a practical embodiment the anastomosis connector has eight staple pins at eight corners of the inner wall, which inner wall is formed as an octagon.

It is noted that numerous alternative shapes of the inner wall and/or the outer wall are conceivable, for example alternative polygons, circular, oval or the like. Similarly, the imaginary circumference of the locations of the staple pins, may have alternative shapes, for example oval or the like.

The inner wall between each pair of neighbouring staple pins in circumferential direction of the annular body may have a recess, wherein the back side of the annular body is provided with a layer of expanded PTFE which also covers the recesses. Expanded PTFE is also known as Gore-Tex. When the anastomosis connector is mounted to a vessel, a surgeon can apply one or more stitches between the vessel and the expanded PTFE at the recesses, if desired. The presence of the recesses provides the surgeon an appropriate view on the locations where the stitches should be applied.

Preferably, the inner wall of the annular body is provided with pairs of notches, wherein the notches of each pair are located at either side of each staple pin, since this appears to provide the opportunity to bend the staple pins without adversely deforming the annular body. Furthermore, it appears that a small bending radius of each of the staple pins at the transition between the annular body and the staple pin can be achieved. This helps the staple pins to clamp the vessel wall against the annular body.

Clamping the vessel wall against the annular body may lead to locally compressing the vessel wall. This may result into protrusion or bulging of tissue into the anastomotic orifice, which increases the above-mentioned effect of tissues which grow to each other over the respective inner walls of two interconnected anastomosis connectors.

The staple pins may be adapted such that they can be bent to predetermined curvatures. The deformation may start at their free ends and progress towards the annular body. This may be achieved by a radial thickness and/or circumferential thickness of each staple pin, which decreases towards its free end. In other words, the staple pins may be tapered along their lengths in radial and/or circumferential direction of the annular body in order to create predetermined bending characteristics.

In a practical embodiment the annular body is plate-shaped. This structure can be manufactured relatively easily. At least the front side may have a substantially flat surface. This provides the opportunity to secure two of such identical anastomosis connectors to each other in a reliable manner with minimum risk of leakage between the abutting front sides.

The staple pins may be substantially linear in their starting position. In their final positions, each staple pin may have a partly circular, substantially circular or oval shape. In general, it may have a non-uniform bending curve in order to create appropriate clamping characteristics of the vessel wall around an opening of the vessel where the anastomosis connector is placed. It is also conceivable that the staple pins have pre-curved free end portions in their starting positions.

The back side of the annular body may have a depressed edge portion along the inner wall such that when the anastomosis connector is mounted to a vessel a portion of the vessel wall may extend in the space that is defined by the depressed edge portion. This provides a stable and substantially leak-free connection. Particularly, in combination with staple pins that are located at the inner wall, the staple pins may clamp a portion of the vessel wall in the mentioned space when they are in their final positions such that tissue of the vessel is locally compressed.

The depressed edge portion may be surrounded by a layer of material that is different from the material of the remainder of the annular body, for example expanded PTFE. The vessel wall around the opening where the anastomosis connector is mounted may grow into the expanded PTFE. The remainder of the annular body or a part thereof may comprise a biocompatible metal, for example.

An advantage of a layer of expanded PTFE at the back side of the annular body, independent from the presence of a depressed edge portion, is that after mounting the anastomosis connector to a vessel a surgeon may create a repair stitch, if necessary, and anchor it in the layer of expanded PTFE.

In a particular embodiment at least a part of the back side of the annular body diverges between the inner wall and the outer wall in a direction from the back side to the front side. This means that in at least the mentioned part the back side crosses a plane which extends parallel to and lies at a distance from a plane in which the inner wall lies. Hence, when the anastomosis connector is mounted to a vessel, the inner wall will be located at the vessel whereas the back side at a distance from the inner wall is also located at a distance from the inner wall in a direction perpendicular to the plane in which the inner wall lies. An advantage of this embodiment is that it can be mounted to a vessel at a location within an anatomically narrow area without being obstructed by its surroundings, for example at a coronary vessel. It is conceivable that the whole back side of the annular body diverges between the inner wall and the outer wall in a direction from the back side to the front side.

The back side may diverge continuously or discontinuously. In case of a continuously diverging back side the back side may be conical.

Of course, the front side must fit to an appropriate cooperating anastomosis connector.

The front side may have the same shape as the back side. For example, the annular body may form a funnel including a small inclination angle, wherein the front side extends parallel to the back side.

The annular body may be provided with a reinforcement portion, which allows a thin structure of the annular body. This appears to be particularly advantageous when the annular body is made of titanium.

The reinforcement portion may comprise a deformed section of the annular body.

In a practical embodiment the deformed section comprises a circular reinforcement ridge surrounding the anastomotic orifice.

Preferably, the reinforcement ridge forms a protrusion at the back side and a depression at the front side of the annular body, since the depression may be used for receiving a sealing ring in order to create a sealed connection with an annular body of another anastomosis connector.

At least one of the inner wall and the back side may be treated by a roughening treatment, such as sandblasting and/or acid treatment or the like, wherein surface roughness of the at least one of the inner wall and the back side may be larger than that of the front side. The increased surface roughness improves tissue attachment to the annular body. Due to this effect the load on the staple pins may decrease after mounting the anastomosis connector to a vessel, which minimizes the risk of material fatigue.

The invention is also related to a group of parts, comprising two anastomosis connectors as described hereinbefore and a fixing member for attaching the annular bodies to each other such that their respective anastomotic orifices are aligned, wherein the front sides of the annular bodies of the respective anastomosis connectors snugly fit to each other.

The fixing member may be a separate part. Nevertheless, it may also be part of one of the anastomosis connectors before mounting them to respective vessels.

The fixing member may be a clamp or glue or the like.

Preferably, the front sides of the annular bodies of the respective anastomosis connectors snugly fit to each other at different mutual angles about centerlines of their anastomotic orifices, since this allows a surgeon to arrange the connected vessels with respect to each other in anatomically favourable orientations.

The fixing member may be adapted to attach the annular bodies releasably to each other. An advantage of this embodiment is that a surgeon can easily detach the anastomosis connectors from each other if, for example, it appears after testing the anastomosis that one of the anastomosis connectors requires a separate treatment such as a re-check or an additional repair stitch without the other anastomosis connector being fixed to it. A final fixation may be performed with a non-releasable fixing member.

The anastomosis connectors may be substantially identical, which is efficient in terms of manufacturing costs.

The upper side of one or both anastomosis connectors may be proved with a fluid tight layer or sealing element, for example silicone or PTFE.

In an embodiment the fixing member is provided with a first coupling element at one of the anastomosis connectors which is insertable in a cooperating first coupling member at the other one of the anastomosis connectors when the annular bodies are angled with respect to each other, wherein the first coupling element and the first coupling member are adapted such that they form a pivot including a pivot axis at the first coupling element and the first coupling member when the first coupling element is received by the first coupling member, and with a second coupling element at said one of the anastomosis connectors which is attachable to a cooperating second coupling member at said other one of the anastomosis connectors, wherein the first coupling element and the second coupling element are located at a distance from each other. This means that the first coupling member and the second coupling member are also located at a distance from each other. They may be located opposite to each other. After inserting the first coupling element of the one anastomosis connector into the first coupling member of the other anastomosis connector their annular bodies can be rotated to each other such that the second coupling element and the second coupling member can be attached to each other.

The first coupling member may have a hook-shaped cross-section or it may comprise a hole.

Preferably, the second coupling element and the second coupling member are attachable to each other through a snapping operation upon rotating the mutually angled annular bodies to each other about the pivot axis, since this facilitates the anastomosis operation. Numerous cooperating snapping elements are conceivable for this type of attachment.

The first coupling element and the first coupling member may be located at the outer wall of the respective annular bodies.

Similarly, the second coupling element and the second coupling member may be located at the outer wall of the respective annular bodies.

The invention is also related to an applicator for mounting an anastomosis connector as described hereinbefore to a flexible vessel at an opening thereof, comprising an elongate element and a pusher which are coupled to each other and movable with respect to each other in a direction along the longitudinal axis of the elongate element, wherein the elongate element has a distal end portion to be put through the anastomotic orifice of the anastomosis connector and to be inserted into the opening of the flexible vessel, wherein the distal end portion is provided with a plurality of anvils protruding in outward direction of the elongate element with respect to a longitudinal axis thereof, which anvils have respective upper sides that are directed to the pusher so as to bend the staple pins under operating conditions when the anastomosis connector is located between the pusher and the anvils and the pusher and the anvils are moved to each other. Under operating conditions the pusher contacts the front side of the anastomosis connector and the staple pins are received by the upper sides of the anvils.

It is noted that herein a distal end refers to an end which is designed for positioning at the location of an anastomosis site and a proximal end refers to an end which is designed for positioning away from the location of the anastomosis site. Correspondingly, a distal direction is defined as a direction from the proximal end to the distal end and a proximal direction is defined as a direction from the distal end to the proximal end.

The anvils may be movable in a transverse direction with respect to the longitudinal axis of the elongate element between retracted positions and extended positions. This means that in the extended positions the anvils surround a larger circumference than in the retracted positions. This embodiment allows to adapt the applicator and the anastomosis connector to each other such that in the extended positions an outer circumference defined by the anvils is larger than the circumference of the inner wall of the annular body, whereas in the retracted positions the anvils can freely pass through the anastomotic orifice. Under operating conditions, the anvils may be extended after being inserted into an opening of a vessel such that the staple pins can contact the anvils and can be bent at the larger circumference.

The pusher may be provided with a holding device, such as a magnet, for temporarily holding an anastomosis connector. This prevents a premature movement of the anastomosis connector towards the anvils which could hinder the view of the surgeon on the location where the anastomosis connector is to be mounted.

In a practical embodiment the pusher surrounds the elongate element such that a uniform force can be exerted onto the annular body of the anastomosis connector.

In a particular embodiment the upper sides of the anvils are concave, which provides the opportunity to bend the staple pins such that their free ends are directed back towards the annular body of the anastomosis connector during movement of the pusher and the anvils to each other. The shape of the upper sides may be partly circular, for example, but alternative shapes to create predetermined bending curves of the staple pins in their final positions are conceivable.

The applicator may be provided with an aligning device for aligning the staple pins of an anastomosis connector with the anvils. This facilitates placing the anastomosis connector to the applicator. For example, the aligning device rotates the anastomosis connector about the elongate element when the anvils and the pusher move to each other such that the staple pins of the anastomosis connector are aligned with the anvils upon contacting each other.

The applicator may be provided with a vessel supporting member for supporting the vessel at a rim of an opening where the anastomosis connector is to be mounted, wherein the vessel supporting member is located at the distal end portion of the elongate member between the pusher and the anvils. This provides the opportunity to create a stable position of the vessel wall for receiving the staple pins during pushing the anastomosis connector and the anvils to each other.

Preferably, the vessel supporting member is expandable in a transverse direction with respect to the longitudinal axis of the elongate element between a retracted position and an expanded position, since from a medical point of view it is desired to stretch the edge of the opening of the vessel before mounting the anastomosis connector to the vessel. Furthermore, this embodiment allows to adapt the applicator and the anastomosis connector to each other such that in the expanded position the circumference of the vessel supporting member is larger than the circumference of the inner wall of the annular body, whereas in the retracted position the vessel supporting member can freely pass through the anastomotic orifice. Hence, the vessel supporting member can be inserted through the anastomotic orifice and the opening of the vessel in its retracted position and subsequently expanded to its expanded position before moving the pusher and the anvils to each other.

After fixing the anastomosis connector to the vessel by moving the pusher and the anvils to each other the vessel supporting member can be set to its retracted position and pulled back through the opening and the anastomotic orifice.

The anvils may extend beyond the vessel supporting member as seen in outward direction of the elongate element when the anvils are in their extended positions and the vessel supporting member is in its expanded position. This provides the opportunity to expand the opening of the vessel to which the anastomosis connector is to be mounted and to bend the staple pins in outward direction of the annular body.

In a practical embodiment the elongate element comprises a shank and a plurality of arms which extend in longitudinal direction of the shank from an end of the shank to free ends of the arms, which arms are movable with respect to the shank in a transverse direction with respect to a longitudinal axis of the shank between retracted positions and extended positions, wherein the distal end portion of the elongate member is formed by distal end portions of the arms, and wherein the applicator is provided with a spreading element which cooperates with the arms and is movable with respect to the arms along the longitudinal axis of the shank, wherein the arms are arranged in circumferential direction about the spreading element and inner sides of the arms and an outer side of the spreading element are adapted such that the arms are moved between their retracted positions and their extended positions upon moving the spreading element with respect to the arms, wherein the vessel supporting member is formed by outer surfaces of the respective arms at the distal end portions thereof and the anvils are also provided at the distal end portions. This is an appropriate mechanical structure to retract and expand the vessel supporting member and the anvils. For example, the arms may be pivotably coupled to the shank or they may be flexible and fixed to the shank so as to allow the movement with respect to the shank. The anvils are located beyond the vessel supporting member as seen from the shank.

The outer surfaces of the arms may be provided with respective recesses extending in longitudinal directions thereof, for receiving free ends of passing staple pins of the anastomosis connector when the pusher and the anvils are moved to each other. When under operating conditions the arms are in their extended positions the opening of the vessel is expanded and the pusher and the anvils can be moved to each other whereas the free ends of the staple pins are received by the recesses such that they pass the opening without penetrating the vessel wall. After the free ends have passed the vessel wall they are bent by the anvils. If the upper sides of the anvils are concave the free ends may be reverted towards the annular body and penetrate the expanded rim of the opening in the vessel wall.

The recesses may also be adapted such that they can guide the staple pins of the anastomosis connector towards the respective anvils.

The spreading element may be mounted to a stem which extends through the shank such that the stem and the shank are movable with respect to each other in their longitudinal directions.

The vessel supporting member, in particular in its expanded position in case of an expandable vessel supporting member, and/or the anvils, particularly in their extended positions in case of extendable anvils, may have different contours, for example circular or oval or the like, in order to apply anastomosis connectors of which the inner wall has a corresponding shape or of which the imaginary circumference of the locations of the staple pins have a corresponding shape. If the staple pins are arranged along an oval contour at the annular body the anvils should form the same contour when the staple pins and the anvils touch each other.

In a particular embodiment the applicator is provided with a holding device for temporarily holding an anastomosis connector to the pusher, wherein the holding device comprises jaws which are movable with respect to the pusher between holding positions for holding an anastomosis connector to the pusher and releasing positions for disconnecting an anastomosis connector from the pusher.

The applicator may have a housing, wherein the jaws may be rotatably mounted to the housing.

The jaws and the pusher may cooperate such that during movement of the pusher towards the anvils the pusher moves the jaws from their holding positions to their releasing positions.

In a preferred embodiment the jaws are fixed to respective bi-stable springs, which are switched from a first stable shape to a second stable shape during movement of the pusher towards the anvils, since this results in a quick de-coupling of the pusher and the anastomosis connector.

In an alternative embodiment the holding device comprises at least a leg between one of the arms and the pusher, which leg is movable with respect to the arm and the pusher in longitudinal direction of the shank, wherein the leg has a free end portion and the arm has an opening, wherein the free end portion has a supporting section for supporting an anastomosis connector, wherein the arm, the leg and the pusher are adapted such that during moving the pusher with respect to the arm in its extended position in a direction towards the anvils the pusher moves the leg in the same direction such that the free end portion slides along the arm and falls into the opening at a predetermined relative position of the arm and the leg. When the free end portion falls into the opening the supporting section is moved towards the arm such that it may be disconnected from a supported anastomosis connector. The pusher and the leg may be adapted such that during moving the pusher towards the anvils the leg may initially be moved together with the pusher until the free end portion falls into the opening of the arm. Under operating conditions the annular body of an anastomosis connector may be sandwiched between the pusher and the supporting section until the free end portion falls into the opening of the arm.

An advantage of the applicator as described hereinbefore is that it can be reloaded after mounting an anastomosis connector to a vessel. Furthermore, the surgeon can have a clear view on proper insertion of the distal portion of the elongate element into a vessel. In case of extendable anvils the surgeon can test the correct location of the anvils within the vessel by slightly pulling the elongate element before moving the pusher including the anastomosis connector mounted to the pusher and the anvils to each other.

The invention is also related to a group of parts, comprising an anastomosis connector and an applicator as described hereinbefore.

The invention is also related to a kit of parts, comprising an applicator and a group of parts comprising two anastomosis connectors as described above.

The invention is also related to the following aspects:

Aspect 1: A method of constructing an anastomosis between two flexible vessels, such as blood vessels, comprising the steps of mounting anastomosis connectors as described hereinbefore to openings in the respective flexible vessels such that the staple pins are fixed to vessel walls of the respective vessels around their openings and the annular bodies are located outside the respective vessels, and attaching the annular bodies of the connectors to each other. An advantage of the method is that each step during creating the anastomosis the surgeon can have full visual control.

Aspect 2: A method according to aspect 1, wherein before and during mounting the anastomosis connectors to the vessels, the respective openings are stretched outwardly.

Aspect 3: A method according to aspect 2, wherein the opening of each vessel is stretched by inserting an expandable vessel supporting member into the vessel and subsequently expanding the vessel supporting member.

Aspect 4: A method according to any one of the aspects 1-3, wherein each of the anastomosis connectors is mounted to the corresponding opening by inserting anvils into the vessel, positioning the staple pins and the anvils in line with each other and pushing the anastomosis connector and the anvils to each other in order to bend the staple pins and pass them through the vessel wall around the opening.

Aspect 5: A method according to aspects 4, wherein the staple pins are initially passed along the vessel wall without penetrating the vessel wall and subsequently bent outwardly such that their free ends revert towards the annular body and penetrate the vessel wall.

### BRIEF DESCRIPTION OF THE DRAWINGS

Aspects of the invention will hereafter be elucidated with reference to very schematic drawings showing embodiments of the invention by way of example.

FIG. 1 is a perspective view of an embodiment of an anastomosis connector, showing staple pins which are in their starting positions.

FIG. 2 is a similar view as FIG. 1, as seen from a different side, showing the staple pins in their final positions.

FIG. 3 is a similar view as FIG. 1, but showing two identical anastomosis connectors that are mounted to respective blood vessels.

FIG. 4 is a similar view as FIG. 3, but showing a condition in which the anastomosis connectors are fixed to each other.

FIGS. 5-7 are similar views as FIG. 1, but showing the anastomosis connector in different conditions.

FIG. 8 is a cross-sectional view of an alternative embodiment of the anastomosis connector.

FIG. 9 is a similar view as FIG. 4, but showing an anastomosis by means of two alternative embodiments of anastomosis connectors.

FIG. 10 is a similar view as FIG. 4, but showing an anastomosis by means of two other alternative embodiments of anastomosis connectors.

FIGS. 11 and 12 are perspective views of an embodiment of a group of parts, illustrating two different embodiments of cooperating anastomosis connectors.

FIG. 13 is a similar view as FIG. 12, showing the group of parts in a fixed condition.

FIG. 14 is an enlarged cross-sectional view of a part of the anastomosis as shown in FIG. 13.

FIGS. 15-20 are perspective views of an embodiment of an applicator including an anastomosis connector, illustrating different conditions thereof.

FIG. 21 is a similar view as FIG. 2, but showing an alternative embodiment of the anastomosis connector.

FIGS. 22-24 are perspective views and side views of an alternative embodiment of a group of parts, illustrating two different embodiments of cooperating anastomosis connectors.

FIGS. 25 and 26 are similar views as FIG. 1, but showing an alternative embodiment of the anastomosis connector as seen from different sides.

FIG. 27 is a cross-sectional view of the embodiment as shown in FIG. 26.

FIG. 28 is a similar view as FIG. 1, but showing an alternative embodiment of the anastomosis connector, which cooperates with the embodiment as shown in FIGS. 25-27.

FIG. 29 is a similar view as FIG. 15, showing an alternative embodiment of the applicator.

FIG. 30 is an enlarged partial view of the applicator as shown in FIG. 29.

FIG. 31 is a similar view as FIG. 30, but showing the applicator in a different condition.

FIG. 32 is an enlarged partial view of FIG. 30, holding the anastomosis connector as shown in FIGS. 25-27.

FIG. 33 is an enlarged partial view of FIG. 30, holding the anastomosis connector as shown in FIG. 28.

FIGS. 34 and 35 are sectional views of the parts as shown in FIGS. 32 and 33, respectively.

FIG. 36 is a similar view as FIGS. 34 and 35, showing the part in a different condition.

FIGS. 37 and 38 are similar views as FIG. 1, but showing an alternative embodiment of the anastomosis connector as seen from opposite sides.

FIGS. 39 and 40 are similar views as FIG. 1, but showing an alternative embodiment of the anastomosis connector as seen from opposite sides, which cooperates with the embodiment as shown in FIGS. 37 and 38.

FIGS. 41 and 42 are enlarged perspective views of the embodiments as shown in FIGS. 37-40, showing successive steps of mounting the cooperating anastomosis connectors to each other.

FIGS. 43 and 44 are similar views as FIG. 15, showing an alternative embodiment of the applicator.

FIG. 45 is an enlarged sectional view of the applicator as shown in FIGS. 43 and 44.

FIG. 46 is a similar view as FIG. 43, showing an alternative embodiment of an applicator.

FIG. 47 is an enlarged sectional view of the applicator as shown in FIG. 46.

### DETAILED DESCRIPTION OF ILLUSTRATIVE EMBODIMENTS

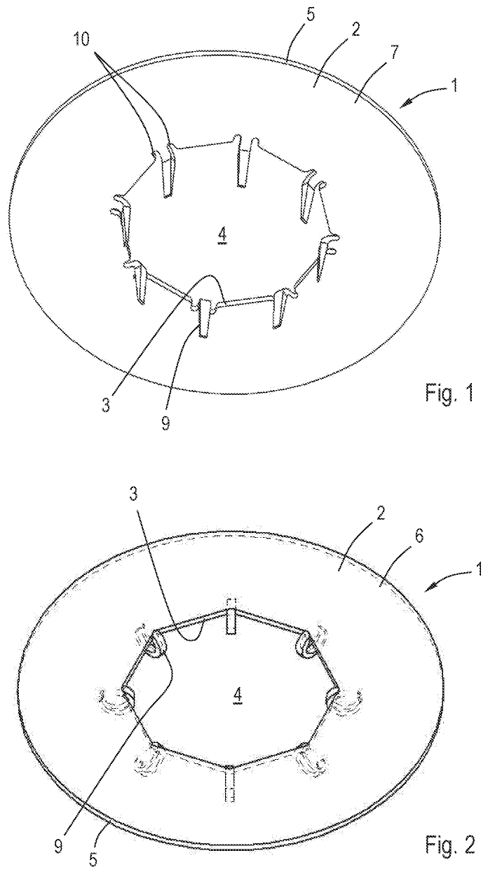

FIGS. 1 and 2 show an embodiment of an anastomosis connector 1. The anastomosis connector 1 is suitable to be mounted to a blood vessel, for example for coronary artery revascularization, which blood vessel may have an inner diameter in the order of magnitude of 2 mm, but it can also be mounted to an alternative flexible vessel. It has a non-expandable annular body 2, which is plate-shaped in this case, extending within a main plane. The anastomosis connector 1 is made of a biocompatible metal which is used in medical technology, for example stainless steel 316LVM or titanium.

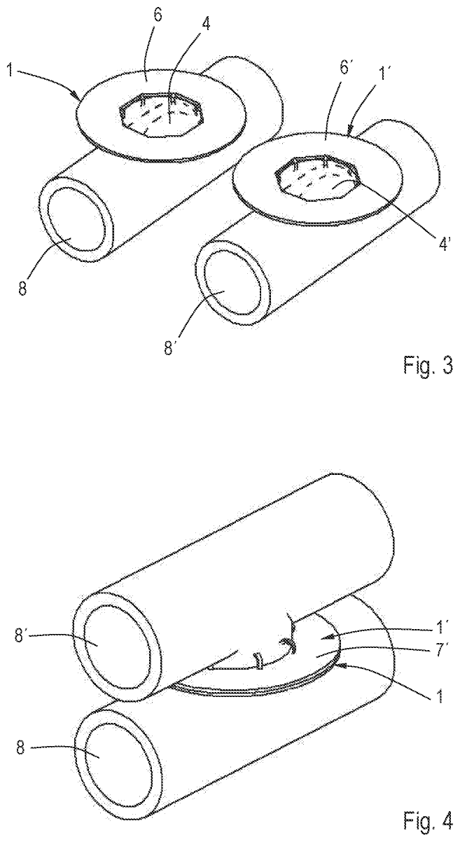

The annular body 2 has an inner wall 3 which surrounds an anastomotic orifice 4, an outer wall 5 which surrounds the inner wall 3, and a front side 6 and a back side 7 which extend between the inner wall 3 and the outer wall 5. In the embodiment as shown in FIGS. 1 and 2 both the front side 6 and the back side 7 have substantially flat surfaces. FIGS. 3 and 4 illustrate how two of such identical anastomosis connectors 1, 1′ are mounted to respective blood vessels 8, 8′ in case of a side-to-side anastomosis, after which they are fixed to each other such that their front sides 6, 6′ are directed to each other. Preferably, the front sides 6, 6′ snugly fit to each other in order to create a fluid tight connection. The respective anastomotic orifices 4, 4′ form a common opening between the blood vessels 8, 8′. There are numerous manners to fix the anastomosis connectors 1, 1′ to each other and examples will be shown hereinafter.

The anastomosis connector 1 as shown in FIGS. 1 and 2 comprises eight staple pins 9 which are located at the inner wall 3 of the annular body 2 at a distance from each other in circumferential direction of the annular body 2. The staple pins 9 project from the annular body 2 and point in a direction away from the back side 7. The anastomosis connector 1 will be fixed to the blood vessel 8 through the staple pins 9.

The inner wall 3 between each pair of neighbouring staple pins 9 in circumferential direction of the annular body 2 extends substantially linearly such that it forms an octagon. The staple pins 9 are located at respective corners of the octagon. When the anastomosis connector 1 is mounted to the blood vessel 8 at an opening thereof, a rim of a blood vessel wall around the opening will extend linearly between each of two neighbouring staple pins 9 that are fixed to the blood vessel wall. As a consequence, the blood vessel wall will follow the linear edge of the inner wall 3, hence minimizing the risk of leakage. The outer wall 5 of the embodiment as shown in FIGS. 1 and 2 forms a circle, but this may be different in an alternative embodiment.

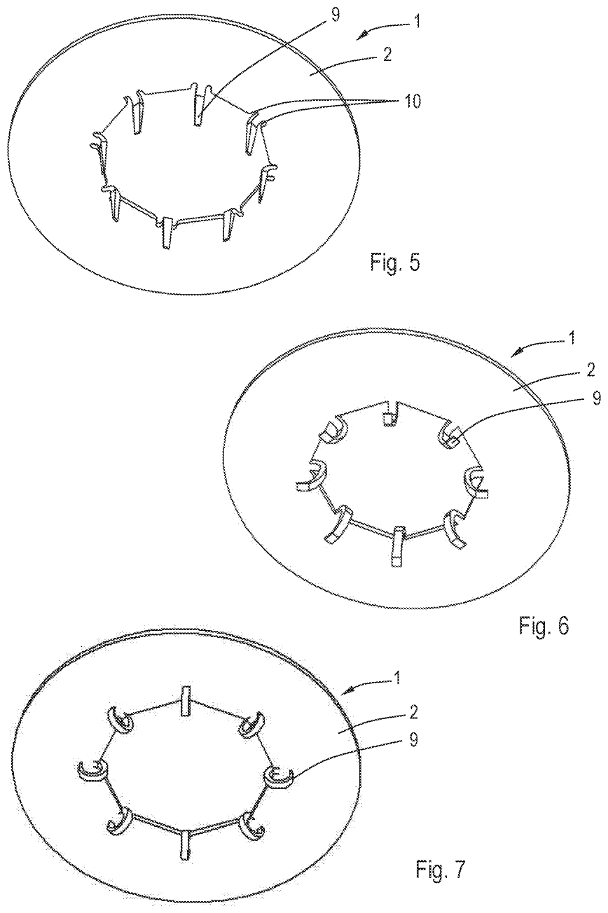

The staple pins 9 of the embodiment as shown in FIG. 1 are configured to be bent permanently from starting positions to final positions in outward direction of the annular body 2, see FIG. 2. The bending process is illustrated in FIGS. 5-7 which successively show the staple pins 9 in the starting positions, intermediate positions and the final positions. In the starting positions the staple pins 9 are substantially linear and extend perpendicularly to the main plane of the annular body 2. In the intermediate positions the staple pins 9 are deformed such that their free ends are directed towards the annular body 2. Upon mounting the anastomosis connector 1 to a blood vessel 8 the staple pins 9 may start to penetrate the blood vessel wall after arriving in the intermediate positions, hence from an inner side of the blood vessel 8. During continued bending the staple pins 9 they will penetrate further into the blood vessel wall and in their final positions they may clamp a portion of the blood vessel wall to the back side 7 of the annular body 2 such that the anastomosis connector 1 is not only fixed to the blood vessel 8, but the annular body 2 and the clamped portion of the blood vessel wall also minimize any leakage between the blood vessel 8 and the annular body 2. FIG. 7 shows that the staple pins 9 have a partly circular shape in their final positions, but they may also have a substantial circular or oval shape, for example.

FIG. 1 shows that the inner wall 3 of the annular body 2 is provided with eight pairs of notches 10. The notches of each pair 10 are located at either side of each staple pin 9 and extend in radial direction from a centerline of the annular body 2 and from the front side 6 to the back side 7. Due to the presence of the pairs of notches 10 the staple pins 9 can obtain small bending radii at their ends which are remote from their free ends.

Referring to FIGS. 3 and 4 it is clear that the inner walls 3, 3′ of the respective annular bodies 2, 2′ will contact blood that flows through the blood vessels 8 after creating the anastomosis. In order to minimize exposure to blood the thickness of the annular bodies 2, 2′ at the respective inner walls 3, 3′ should be small. More specifically, the distance between the front side 6 and the back side 7 of the annular body 2 is smaller than the distance between the inner wall 3 and the outer wall 5. In order to create sufficient material to connect the two anastomosis connectors 1 to each other the distance between the inner wall 3 and the outer wall 5 is larger than the thickness of any of the staple pins 9 as measured in a direction from the inner wall 3 to the outer wall 5 at the location of the staple pin 9.

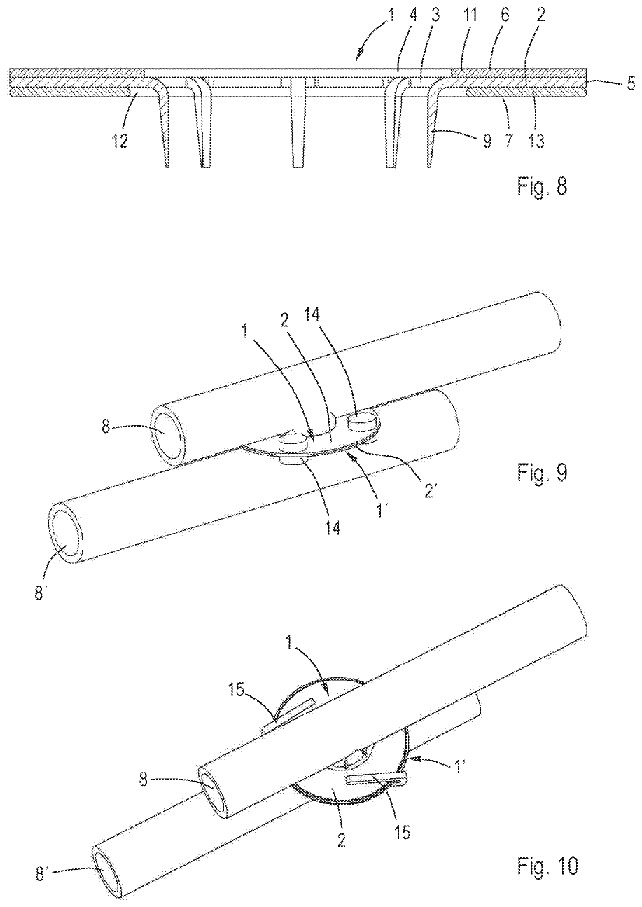

FIG. 8 shows an alternative embodiment of the anastomosis connector 1. In this embodiment the upper side 6 of the annular body 2 is provided with a sealing layer 11 which forms a seal between two anastomosis connectors 1 that are fixed to each other. The sealing layer 11 may be a silicone coating for example. The back side 7 of the annular body 2 has a depressed edge portion 12 along the inner wall 3. The depressed edge portion 12 is surrounded by a layer of expanded PTFE 13, also known as Gore-Tex; the porosity of this material permits the body's own tissue to grow into the material and it is nearly inert inside the body. During mounting the anastomosis connector 1 to the blood vessel 8 a portion of the blood vessel wall around the opening of the blood vessel 8 can be clamped in the depressed edge portion 12, whereas the vessel wall can grow into the layer of expanded PTFE 13. Although in FIG. 8 the sealing layer 11 and the layer of expanded PTFE 13 have more or less the same thickness as a core of the annular body 2 which is sandwiched between them, the sealing layer 11 should be as thin as possible whereas the expanded layer of PTFE 13 may be thicker than the core of the annular body 2 in order to provide sufficient material for applying additional stitches between a vessel 8 to which the anastomosis connector 1 is mounted and the layer of expanded PTFE, if desired.

FIG. 21 shows an alternative embodiment of an anastomosis connector 1, wherein the inner wall 3 between each pair of neighbouring staple pins 9 in circumferential direction of the annular body 2 has a recess 14. The recesses 14 are located beyond the staple pins 9 as seen from a center of the anastomotic orifice 4. The back side 7 of the annular body 2 is provided with a layer of expanded PTFE 13 which also covers the recesses 14 and which is fixed to the core of the annular body 2. The spaces which are formed by the recesses 14 may be filled with expanded PTFE, as well. In the embodiment as shown in FIG. 21 the inner wall 3 at the recesses 14 has a partly circular profile, but numerous alternative shapes are conceivable. An advantage of this embodiment is that it enables a surgeon to construct additional stitches between a vessel 8 to which the anastomosis connector 1 is mounted and the layer of expanded PTFE between each pair of neighbouring staple pins 9.

FIG. 9 shows two alternative embodiments of anastomosis connectors 1, 1′ which are each provided with four magnets 14 for releasably attaching the annular bodies 2, 2′ to each other in a side-to-side anastomosis of two blood vessels 8, 8′. The magnets 14 are fixed to the annular bodies 2, 2′, for example by means of glue. They may be coated by a biocompatible material, for example gold. The magnets 14 are located such that the annular bodies 2, 2′ can be fixed to each other at four different angles about common centerlines of the annular bodies 2, 2′. In fixed condition the anastomotic orifices 4, 4′ of the respective annular bodies 2, 2′ are aligned.

In the anastomosis as shown in FIG. 9 the two identical anastomosis connectors 1, 1′ including the magnets 14 form an embodiment of a group of parts having two anastomosis connectors 1, 1′ and a fixing member, wherein the fixing member is formed by the magnets 14. Since the magnets 14 are fixed to the annular bodies 2, the fixing member is integrated in the respective anastomosis connectors 1, 1′. Since the respective anastomosis connectors 1, 1′ are applied in the blood vessels 8, 8′, the magnetic force must be such that the magnets 14 can withstand blood pressure between the annular bodies 2, 2′. An advantage of applying the magnets 14 is that the annular bodies 2, 2′ are instantaneously coupled to each other upon approaching each other, which facilitates endoscopic application.

The fixing member may also be a separate part, which is illustrated in the embodiment of a group of parts that are fixed to each other as shown in FIG. 10. In this case the annular bodies 2, 2′ are attached to each other through two clamps 15. The front sides 6, 6′ of the annular bodies 2, 2′ and the anastomotic orifices 4, 4′ are adapted such that the annular bodies 2, 2′ snugly fit to each other at any desired angle about common centerlines of the annular bodies 2, 2′. In case of circular inner walls 3, 3′ they can remain aligned exactly.

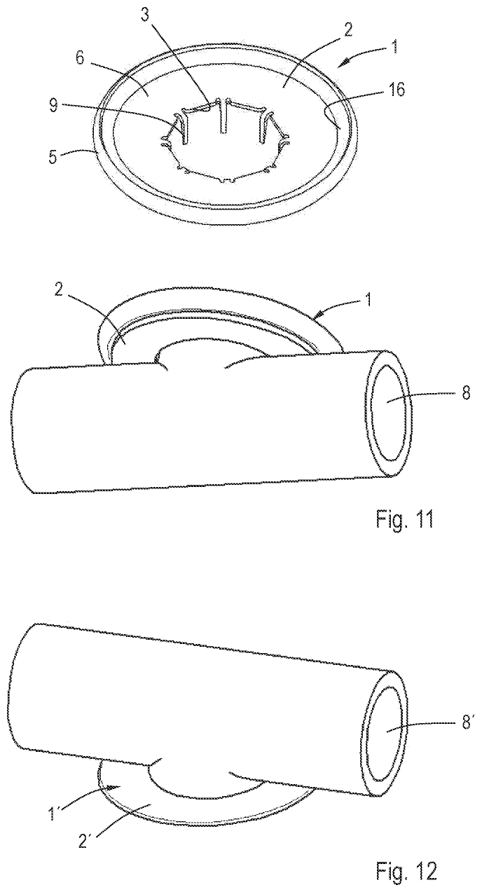

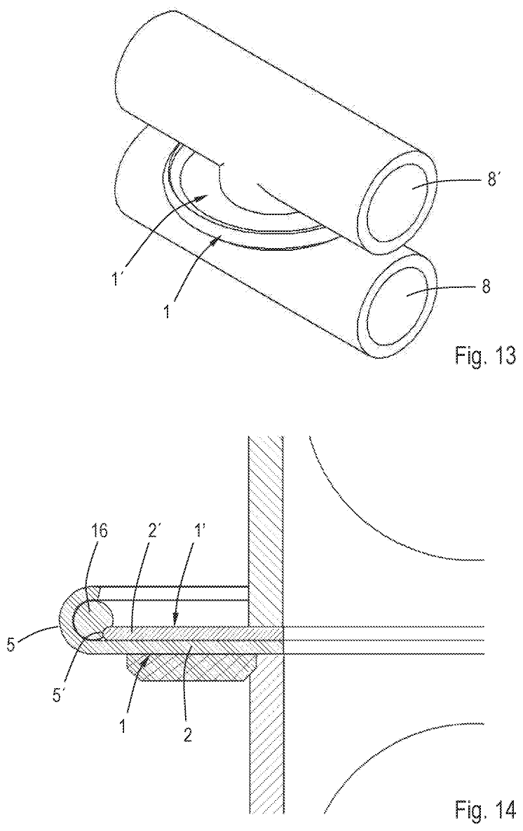

FIGS. 11-14 show another alternative group of parts, wherein the annular bodies 2, 2′ of two alternative anastomosis connectors 1, 1′ are different. Both annular bodies 2, 2′ are plate-shaped, but one of them has an upright curved edge at its outer wall 5 which curved edge is directed away from its front side 6, whereas the other one has an entirely flat front side 6′. A flexible ring 16 is located inside the curved edge. The anastomosis connectors 1, 1′ can be attached to each other by pushing the annular bodies 2, 2′ to each other such that the flat annular body 2′ passes the flexible ring 16; the resulting condition is shown in FIGS. 13 and 14. It is noted that numerous alternative snap fittings between the anastomosis connectors 1, 1′ are conceivable.

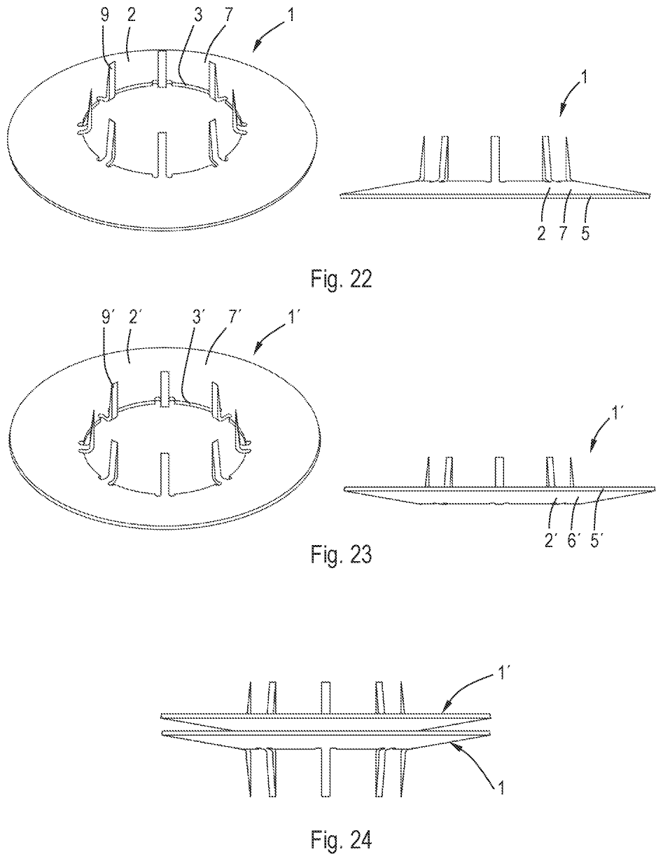

FIGS. 22-24 show still another alternative group of parts, wherein the annular bodies 2, 2′ of two alternative cooperating anastomosis connectors 1, 1′ are also different. Both annular bodies 2, 2′ have a conical shape. The annular body 2 of the anastomosis connector 1 as shown in FIG. 22 diverges from the inner wall 3 to the outer wall 5 in a direction from the back side 7 to the front side 6. The annular body 2′ of the anastomosis connector 1′ as shown in FIG. 23 diverges from the inner wall 3′ to the outer wall 5′ in a direction from the front side 6′ to the back side 7′. An advantage of the shape of the annular body 1 as shown in FIG. 22 is that it is appropriate to be mounted to a blood vessel 8 at a location within an anatomically narrow area, for example a location which is surrounded by other blood vessels or organs. In such a case the diverging annular body 2 can be mounted to the blood vessel 8 without being obstructed or obstructing the surroundings. The annular body 2′ of the cooperating anastomosis connector 1′ as shown in FIG. 23 fits to the annular body 2 of the anastomosis connector 1′ as shown in FIG. 22. This is illustrated in FIG. 24. Due to the conical shapes of the cooperating annular bodies 2, 2′ they are self-centering. Besides, they fit to each other at different mutual angles about centerlines of their anastomotic orifices 4, 4′.

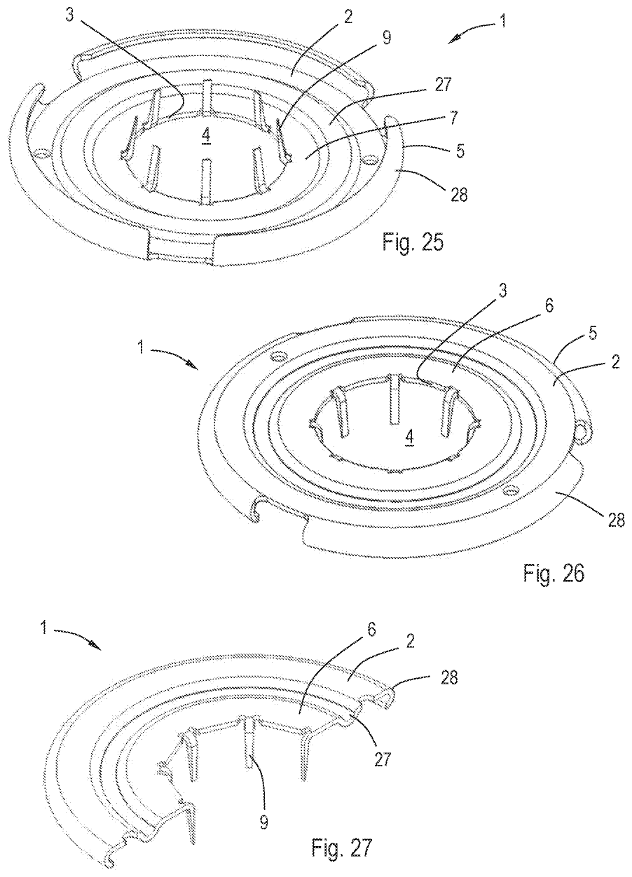

FIGS. 25-27 show different views of an alternative embodiment of an anastomosis connector 1. The same reference numbers as used in earlier embodiments for corresponding elements are applied. The non-expandable annular body 2 is plate-shaped, but between the inner wall 3 and the outer wall 5 the annular body 2 is provided with a circular reinforcement ridge 27. The reinforcement ridge 27 comprises a deformed portion of the annular body 2 and surrounds the anastomotic orifice 4. In this case the reinforcement ridge 27 forms a protrusion at the back side 7 and a depression at the front side 6 of the annular body 2. The reinforcement ridge 27 creates a rigid anastomosis connector 1 and allows a thin annular body 2, which minimizes contact with blood after creating the anastomosis. Particularly, the reinforcement ridge 27 is advantageous if the anastomosis connector 1 is made of titanium. Furthermore, the depression at the front side 6 of the annular body 2 provides the opportunity to receive a sealing ring. The anastomosis connector 1 has an upright curved rim 28 at its outer wall 5 which rim 28 is directed away from its back side 7, i.e. in the same direction as the staple pins 9, and which has a convex outer surface. It is noted that the annular body 2 may be provided with an alternative reinforcement rather than the circular reinforcement ridge 2.

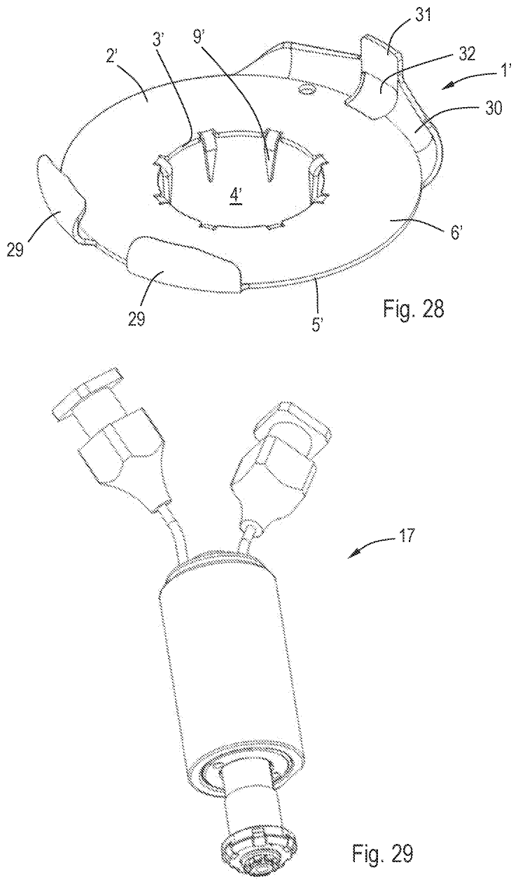

FIG. 28 shows an alternative embodiment of an anastomosis connector 1′, which can cooperate with the anastomosis connector 1 as shown in FIGS. 25-27 so as to create an anastomosis. This means that the anastomosis connector 1 as shown in FIGS. 25-27 and the anastomosis connector 1′ as shown in FIG. 28 may form an embodiment of a group of parts. Referring to FIG. 28, the annular body 2′ of the anastomosis connector 1′ is plate-shaped having a flat front side 6′. In a fixed condition of the cooperating anastomosis connectors 1, 1′ their front sides 6 and 6′ may contact each other outside the reinforcement ridge 27 of the anastomosis connector 1 as shown in FIGS. 25-27. A secure sealing between the cooperating anastomosis connectors 1, 1′ may be achieved by applying a sealing ring in the depression at the front side 6 of the annular body 2.

The anastomosis connector 1′ as shown in FIG. 28 comprises a pair of first coupling members 29 and one second coupling member 30 at the outer wall 5′ of the annular body 2′. The first and second coupling members 29, 30 form curved projections which project from the front side 6′ and which have concave inner surfaces such that they have hook-shaped cross-sections. The curvature is such that the rim 28 of the anastomosis connector 1 as shown in FIGS. 25-27 fits within the first and second coupling members 29, 30. In other words, the convex outer surface of the rim 28 matches with the concave inner surfaces of the first and second coupling members 29, 30. The height of the pair of first coupling members 29 as measured from the front side 6′ is larger than the height of the second coupling member 30. A permanently deformable locking element 31 is provided at an upper side of the second coupling member 30.

In practice, the anastomosis connectors 1, 1′ as shown in FIGS. 25-28 can be attached to each other as follows. After a surgeon has fixed the separate anastomosis connectors 1, 1′ to respective blood vessels the anastomosis connector 1′ as shown in FIG. 28 is connected to the anastomosis connector 1 as shown in FIGS. 25-27 by inserting the rim 28 of the anastomosis connector 1 into the spaces defined within the concave inner surfaces of the first coupling members 29 when the respective annular bodies 2, 2′ are angled with respect to each other. In this condition the first coupling members 29 and the rim 28 form a pivot including an imaginary pivot axis. Subsequently, the annular bodies 2, 2′ can be rotated with respect to each other about the pivot axis until the rim 28 snaps into the space defined within the concave inner surface of the second coupling member 30. In the latter condition the respective front sides 6, 6′ of the annular bodies 2, 2′ contact each other. In this case a portion of the rim 28 forms a first coupling element which cooperates with the first coupling members 29 and another portion of the rim 28 forms a second coupling element which cooperates with the second coupling member 30.

After snapping the anastomosis connectors 1, 1′ to each other the surgeon can check whether the resulting anastomosis has been created correctly. If not, an aperture 32 in the second coupling member 30 allows the surgeon to insert a stabbing tool (not shown) between the respective front sides 6, 6′ in order to lift the anastomosis connectors 1, 1′ with respect to each other at the second coupling member 30 and release the rim 28 of the anastomosis connector 1 from the second coupling member 30. Then, the rim 28 of the anastomosis connector 1 can be removed from the first coupling members 29, as well. If the resulting anastomosis has been created correctly, the surgeon can bend the locking element 31 over the rim 28 in order to create a secured lock of the anastomosis connectors 1, 1′.

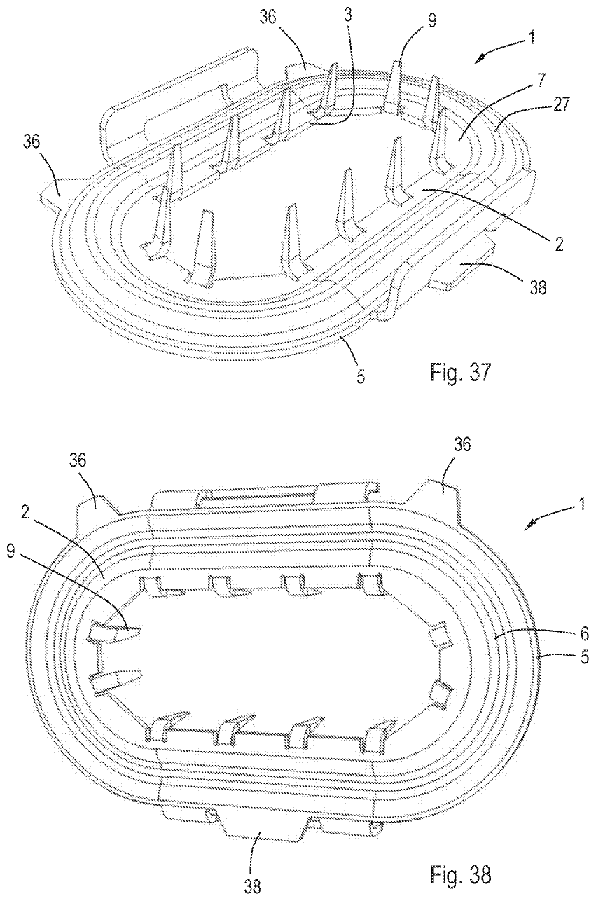

FIGS. 37-42 show alternative embodiments of anastomosis connectors 1, 1′, which have oval-shaped annular bodies 2, 2′. Same reference numbers as used in earlier embodiments for corresponding elements are applied. The anastomosis connectors 1, 1′ can cooperate with each other to create an anastomosis, for example a side-to-side anastomosis of two blood vessels. The anastomosis connector 1 as shown in FIGS. 37 and 38 is provided with a reinforcement ridge 27.

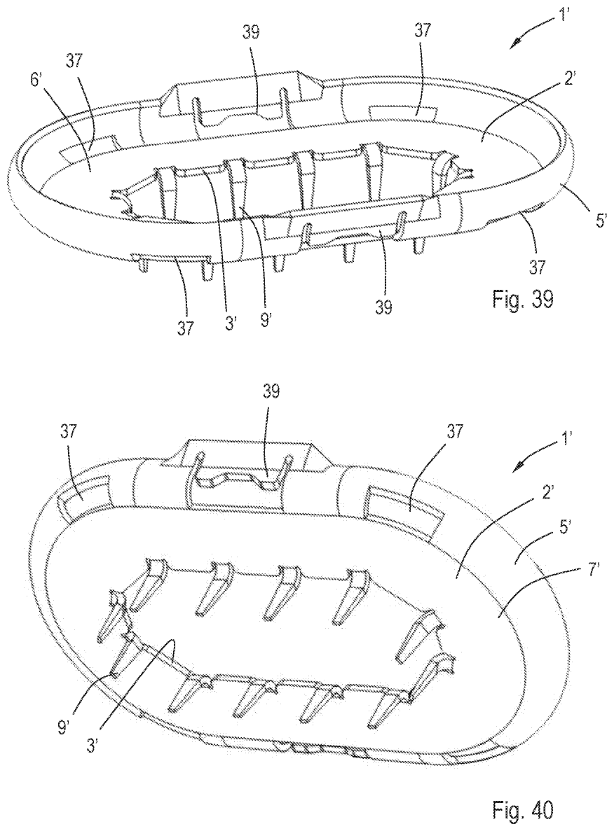

The anastomosis connector 1 as shown in FIGS. 37 and 38 comprises a pair of first ears 36 which fit in one pair of two pairs cooperating through-holes 37 in the anastomosis connector 1′ as shown in FIGS. 39 and 40. Furthermore, it comprises a second ear 38 which can be snapped behind one of two opposite flexible lips 39 at the anastomosis connector 1′ as shown in FIGS. 39 and 40. The latter anastomosis connector 1′ is symmetrical with respect to a central plane between the opposite flexible lips 39 such that the anastomosis connector 1 as shown in FIGS. 37 and 38 can be fixed to it at two different positions.

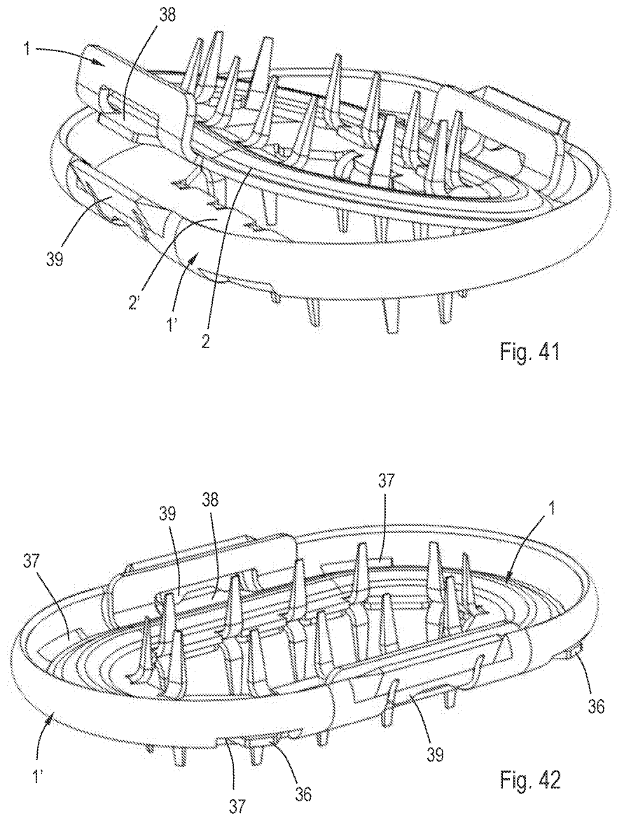

FIGS. 41 and 42 show how the anastomosis connectors 1, 1′ as shown in FIGS. 37-40 can be attached to each other. The anastomosis connector 1 is connected to the anastomosis connector 1′ by inserting the first ears 36 ears into one of the cooperating pairs of through-holes 37 when the respective annular bodies 2, 2′ are angled with respect to each other. Subsequently, the annular bodies 2, 2′ can be rotated with respect to each other about an imaginary pivot axis at the through-holes 37 until the second ear 38 snaps behind the lip 39. In the latter condition the respective front sides 6, 6′ contact each other. It is noted that the first ears 36 and the cooperating through-holes 37 are functionally comparable to the rim 28 and the cooperating first coupling members 29 of the anastomosis connectors 1, 1′ as shown in FIGS. 25-28. Similarly, the second ear 38 and the cooperating lip 39 are functionally comparable to the rim 28 and the cooperating second coupling member 30 of the anastomosis connectors 1, 1′ as shown in FIGS. 25-28. In this case the first ears 36 form a first coupling element and the second ear 38 forms a second coupling element, whereas each pair of through-holes 37 forms a first coupling member and each of the flexible lips 39 forms a second coupling member.

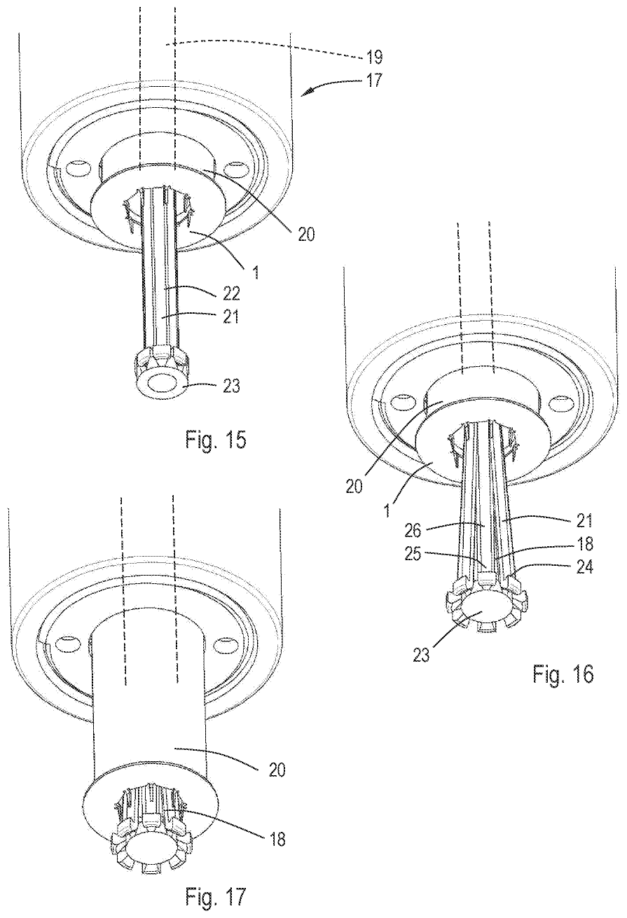

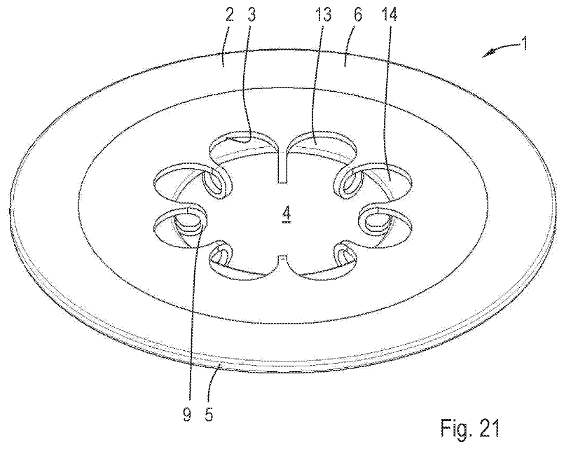

FIGS. 15-20 show an embodiment of an applicator 17 for mounting an anastomosis connector 1 to a blood vessel 8 at an opening thereof. The applicator 17 has a central stem 18 which is visible in FIGS. 16 and 17. The central stem 18 is mounted inside a shank 19 and movable within the shank 19 in longitudinal direction thereof. A pusher 20 is mounted about the shank 19 and movable with respect to the shank 19 in longitudinal direction thereof. A distal end of the shank 19 is provided with flexible arms 21 including free distal ends. The arms 21 are movable in radial direction with respect to a longitudinal axis of the shank 19. In this case the shank 19 and the arms 21 are made from a rigid tube wherein the arms 21 are created by cutting parallel slots 22 which are located at angular distance from each other in circumferential of the tube and extend from the distal end of the tube to a location towards a proximal end thereof, see FIG. 15. Hence, distal ends of the arms 21 are located at the distal end of the tube. In an alternative embodiment the flexible arms 21 may be separate elements which are fixed to a shank or the arms may be flexible or rigid elements that are pivotable with respect to the shank 19.

A distal end of the central stem 18 is provided with a spreading element 23 in the form of a thickened end portion. The distal ends of the arms 21 are arranged in circumferential direction about the spreading element 23. Inner sides of the arms 21 and a cooperating outer side of the spreading element 23 are adapted such that the arms 21 are moved outwardly upon moving the central stem 18 including the spreading element 23 with respect to the shank 19 in a direction from its distal end towards its proximal end. The spreading element 23 has guide grooves for guiding the distal ends of the arms 21 such that they follow a predetermined path. The spreading element 23 and the arms 21 as shown in FIGS. 15-20 have substantially circular contours, but they may have a different shape, for example oval, in order to apply anastomosis connectors 1 which have different shapes.

FIG. 15 shows a condition in which the spreading element 23 does not move the arms 21; the arms 21 are in a retracted position, which means that they are aligned with the shank 19 in this case. FIG. 16 shows a condition in which the central stem 18 is lifted with respect to the shank 19 in a direction from its distal end towards its proximal end such that the arms 21 move outwardly with respect to the longitudinal axis of the shank 19.

The free ends of the arms 21 are provided with respective anvils 24 which project in outward direction of the arms 21. The number of anvils 24 equals the number of staple pins 9 of the anastomosis connector 1 to be mounted to a blood vessel 8. The anvils 24 have concave upper sides which are directed to the pusher 20. The concave upper sides may be partly circular, for example. The anvils 24 are suitable for bending the staple pins 9 of the anastomosis connector 1 when the pusher 20 pushes the anastomosis connector 1 to the anvils 24.

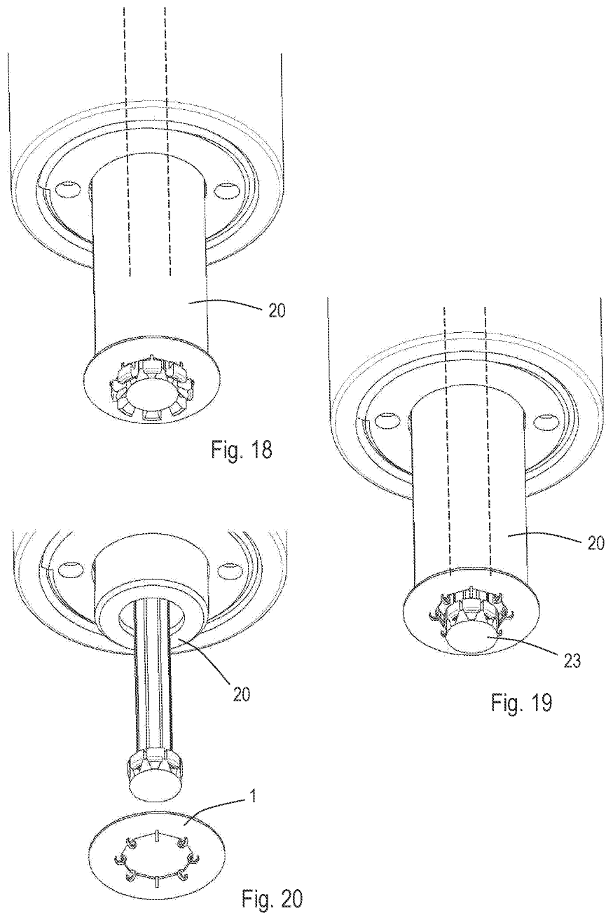

The pusher 20 is provided with a holding device (not shown), such as a magnet or easily releasable material, for temporarily holding an anastomosis connector 1. This prevents the anastomosis connector 1 from unintendedly and prematurely moving towards the anvils 24 and provides a surgeon visual control on the arms 21 when mounting the anastomosis connector 1 to a blood vessel 8. FIGS. 17 and 18 show successive situations during moving the pusher 20 and the anastomosis connector 1 in distal direction to the anvils 24. In the situation as shown in FIG. 19 the central stem 18 is moved with respect to the shank 19 in a direction from its proximal end to its distal end such that the arms 21 return to their retracted positions. This allows the applicator 17 to be removed from an anastomosis site by pulling the end portions of the retracted arms 21 through the anastomotic orifice 4. The holding device of the pusher 20 is adapted such that the pusher 20 can be removed easily from the annular body 2 of the anastomosis connector 1.

A surgeon who uses the applicator 17 to mount an anastomosis connector 1 to a blood vessel 8 may first create an opening in the vessel wall by an incision at a location where the anastomosis connector 1 should be mounted. Subsequently, the applicator 17 including the anastomosis connector 1 in the condition as shown in FIG. 15 will be moved towards the blood vessel 8 and the free ends of the arms 21 and the spreading element 23 will be passed through the opening such that the spreading element 23, the anvils 24 and sections of the arms 21 that are adjacent to the anvils 24 are located inside the blood vessel 8. Subsequently, the spreading element 23 will be lifted with respect to the shank 19 as shown in FIG. 16. Preferably, the opening in the blood vessel 8 is such that the arms 21 will expand the blood vessel wall around the opening during moving outwardly. For that reason, outer surfaces of the respective sections of the arms 21 that are adjacent to the anvils 24 which contact the blood vessel wall around the opening form a vessel supporting member 25, see FIG. 16. It is noted that the anvils 24 are also provided at the arms 21, but they are located beyond the vessel supporting member 25 as seen from the shank 19.

After stretching the blood vessel wall around the opening, the surgeon moves the pusher 20 including the anastomosis connector 1 in distal direction towards the anvils 24. Before the staple pins 9 reach the anvils 24 they pass the expanded opening in the blood vessel 8 without penetrating the vessel wall. This is achieved by the presence of recesses 26 in the outer surfaces of the respective arms. The recesses 26 extend in longitudinal directions of the arms 26 and provide space for the free ends of the staple pins 9 to pass the expanded opening in the blood vessel 8 without penetrating the vessel wall. The recesses 26 may be adapted such that they guide the staple pins 9 of the anastomosis connector 1 towards the respective anvils 24.

When the staple pins 9 are pressed against the anvils 24 they will be bent and penetrate the blood vessel around the expanded opening after their free ends are reverted and face the annular body 2 of the anastomosis connector 1. After the anastomosis connector 1 is fixed to the blood vessel 8 the surgeon can move the central stem 18 in distal direction with respect to the shank 19 such that the spreading element 23 and the free ends of the arms 21 can easily pass the anastomotic orifice 4 of the annular body 2 upon removing the applicator 17.

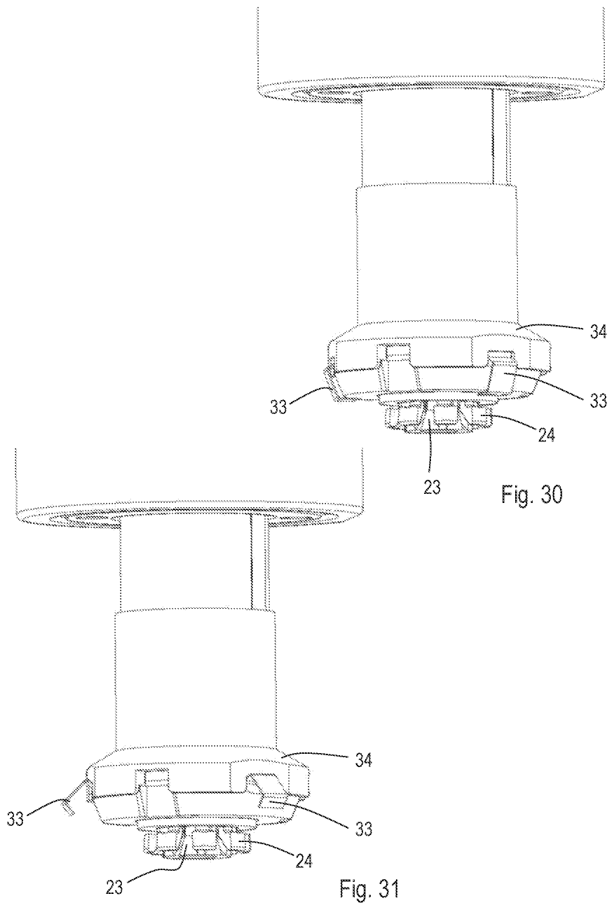

FIG. 29 shows an alternative embodiment of the applicator 17 which is suitable for mounting the different anastomosis connectors 1, 1′ as shown in FIGS. 25-28 to respective blood vessels. FIGS. 30-36 show the applicator 17 in more detail. Elements which correspond to elements in the embodiment as shown in FIGS. 15-20 have the same reference numbers. The applicator 17 is provided with movable arms including anvils 24 and the spreading element 23 for moving the anvils 24 in outward direction upon moving it with respect to the arms in a direction from its distal end to its proximal end. The applicator 17 comprises a pusher for pushing the anastomosis connectors 1, 1′ to the anvils 24, which pusher is not shown for clarity reasons. The spreading element 23, the anvils 24 and the pusher are functionally comparable to the embodiment as shown in FIGS. 15-19

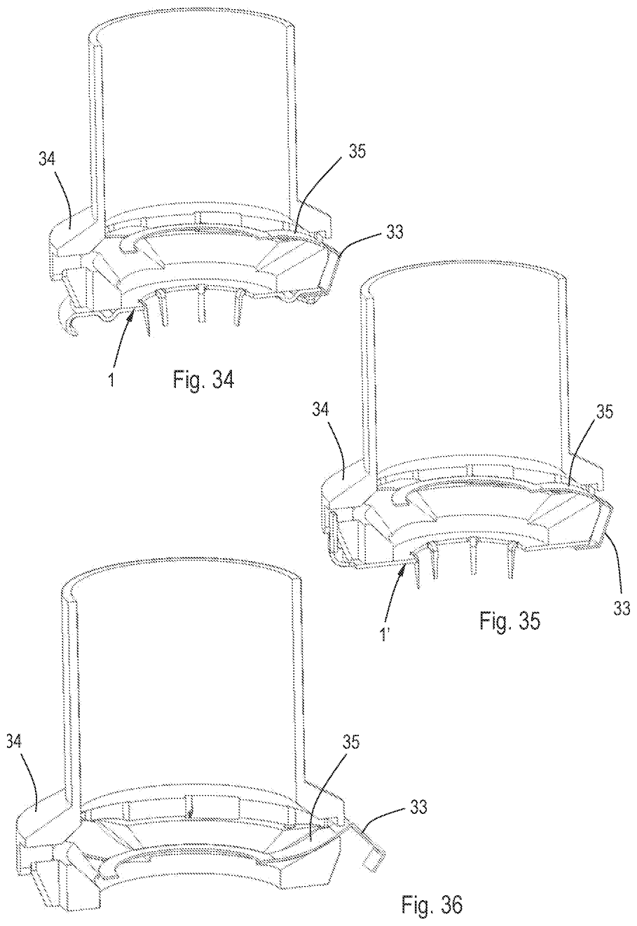

The applicator 17 is provided with a holding device in the form of jaws 33 for temporarily holding an anastomosis connector 1, 1′. In this case the applicator 17 has three jaws 33 which are located at equiangular distance from each other in circumferential direction of a collar 34, see FIGS. 34-36. The collar 34 forms part of a housing of the applicator 17. The jaws 33 are rotatably mounted to the collar 34. They are fixed to respective bi-stable springs 35 which in turn are fixed to a central ring from which the bi-stable springs 35 project in radial outward direction.

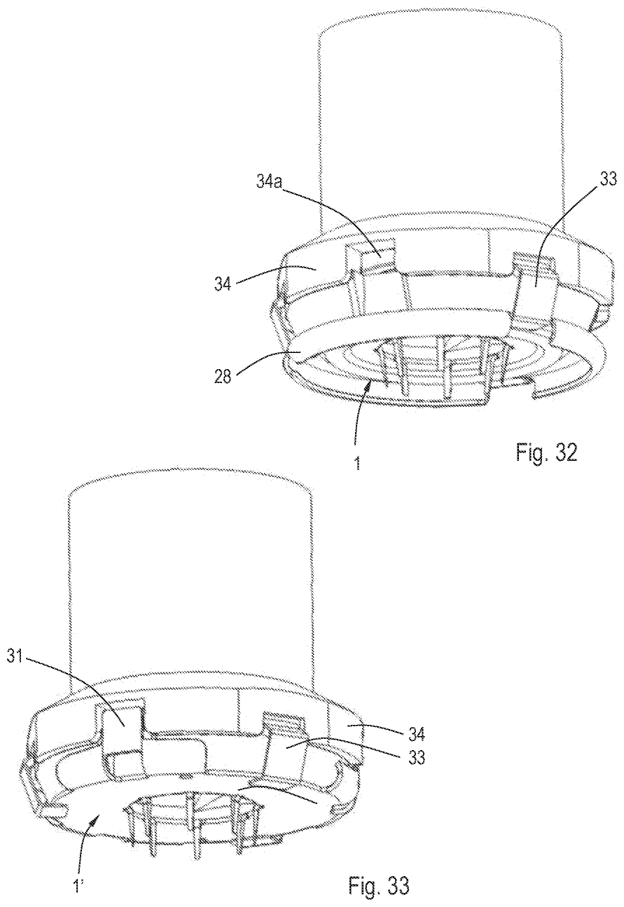

FIGS. 32 and 34 show how the jaws 33 hold the anastomosis connector 1 as shown in FIGS. 25-27. It can be seen that the rim 28 of the anastomosis connector 1 comprises corresponding openings to allow the jaws 33 to contact the back side 7 of the anastomosis connector 1. FIGS. 33 and 35 show how the jaws 33 hold the anastomosis connector 1′ as shown in FIG. 28. It can be seen that the collar 34 is shaped such that it can receive the projecting first and second coupling members 29, 30 and the locking element 31 of the anastomosis connector 1′ as shown in FIG. 28. FIGS. 32 and 33 illustrate that the locking element 31 can be received in a cooperating recess 34a of the collar 34. This means that the applicator 17 is universal for applying both different anastomosis connector 1, 1′, which is very efficient.

FIG. 36 illustrates a situation in which the jaws 33 are moved outwardly to release the anastomosis connectors 1, 1′ from the applicator 17. When comparing the shape of the spring 35 between FIG. 36, on the one hand, and FIGS. 34 and 35, on the other hand, it can be seen that the bi-stable spring 35 between the central ring and one of the jaws 33 varies between opposite curved shapes, i.e. a first stable shape and a second stable shape, corresponding to holding positions and releasing positions of the jaws 33. Using bi-stable springs provides a quick release of the jaws 33 upon moving the central ring with respect to the collar 34 in distal direction, i.e. from the position as shown in FIGS. 34 and 35 to the position as shown in FIG. 36.

Referring to FIGS. 34 and 35 the pusher (which is not shown in FIGS. 29-36) is located within the collar 34 and movable with respect to the collar 34. It is shaped such that it contacts the front sides 6, 6′ of the respective anastomosis connectors 1, 1′ close to their anastomotic orifices 4, 4′ only. Furthermore, the pusher is shaped such that upon contacting the front sides 6, 6′, or just before or after that moment, it also contacts the central ring at the springs 35. Consequently, upon forcing the staple pins 9 and the anvils 24 to each other the jaws 33 will automatically open and release the corresponding anastomosis connector 1, 1′. As a result, after bending the staple pins 9 and retracting the anvils 24 the applicator 17 can be separated from the applied anastomosis connector 1, 1′.

The applicator 17 and the different anastomosis connectors 1, 1′ form an embodiment of a kit of cooperating parts. The applicator 17 is applicable for both types of anastomosis connectors 1, 1′. The locations of the jaws 33, the shape of the collar 34 and the shapes of the anastomosis connectors 1, 1′ are selected such that under operating conditions the anvils 24 are aligned with the staple pins 9.

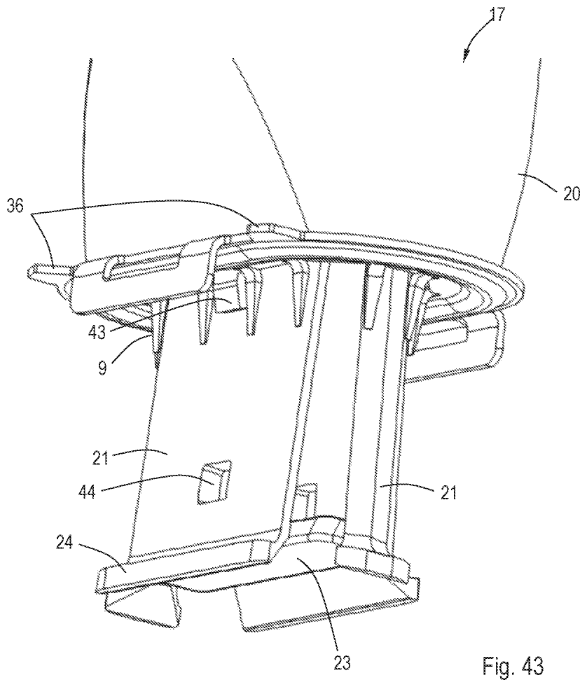

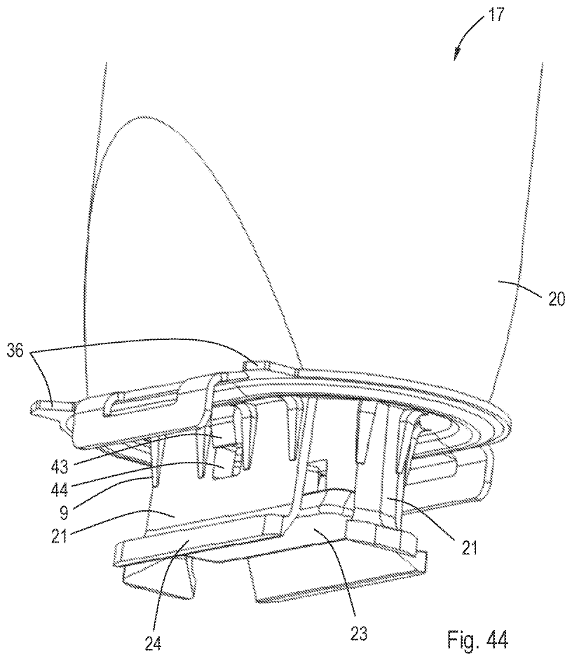

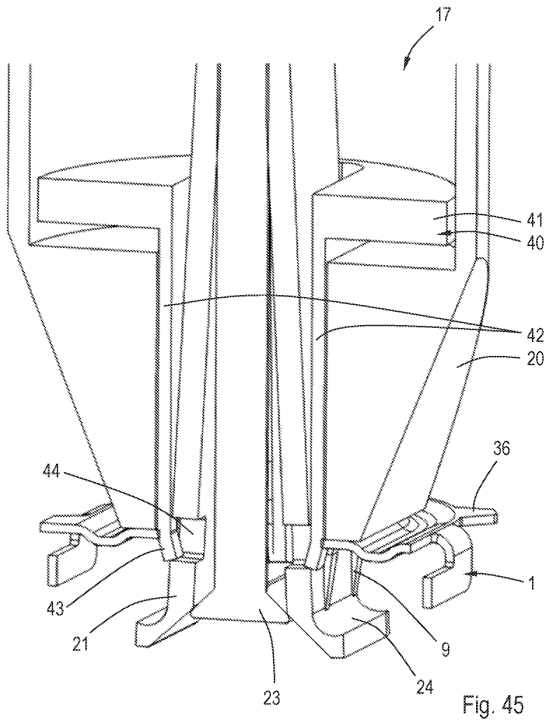

FIGS. 43-45 show parts of an alternative embodiment of the applicator 17 which is suitable for mounting the different oval-shaped anastomosis connectors 1, 1′ as shown in FIGS. 37-40 to respective blood vessels. Elements which correspond to elements in the embodiment as shown in FIGS. 15-20 or FIGS. 29-36 have the same reference numbers. Similar to the earlier embodiments the applicator 17 is provided with movable arms 21 including anvils 24 and a spreading element 23 for moving the anvils 24 in outward direction, but their shapes are different in order to fit to the oval-shaped anastomosis connectors 1, 1′. The applicator 17 also comprises a pusher 20 for pushing the anastomosis connectors 1, 1′ to the anvils 24.

The applicator 17 is provided with a holding device 40 for temporarily holding an anastomosis connector 1, 1′. The holding device 40 has a disk 41 from which two opposite flexible legs 42 project towards the anvils 24. The legs 42 have free end portions 43 which are slightly bent inwardly such that sections of the legs 42 above the respective bends can support the annular body 2, 2′ of a corresponding anastomosis connector 1, 1′. Under operating conditions the free end portions 43 cooperate with openings 44 in the respective arms 21 as follows. When the pusher 20 moves towards the anvils 24 the disk 41 may initially lie on an upper surface of the pusher 20 and move together with the pusher 20 such that the legs 42 also move along the spread arms 21 towards the anvils 24, while keeping the annular body 2, 2′ of a corresponding anastomosis connector 1, 1′ against the pusher 20. The pusher 20, the arms 21 and the legs 42 are shaped such that at a predetermined relative position of the pusher 20, the arms 21 and the legs 42 the free end portions 43 fall into the respective openings 44 of the arms 21 such that they move inwardly and no longer support the corresponding anastomosis connector 1, 1′. When moving the pusher 20 further towards the anvils 24 the free end portions 43 may touch lower edges of the respective openings 44 such that the holding device 40 cannot move further towards the anvils 24 which ensures that the corresponding anastomosis connector 1, 1′ is released. The legs 42 may be resilient such that during movement of the free end portions 43 along the arms 21 the legs 42 are resiliently moved outwardly and bounce back as soon as the free end portions 43 arrive at the respective openings 44.

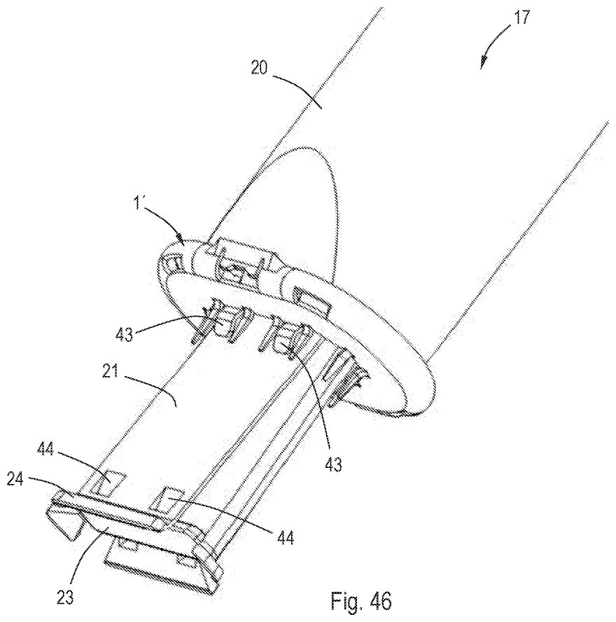

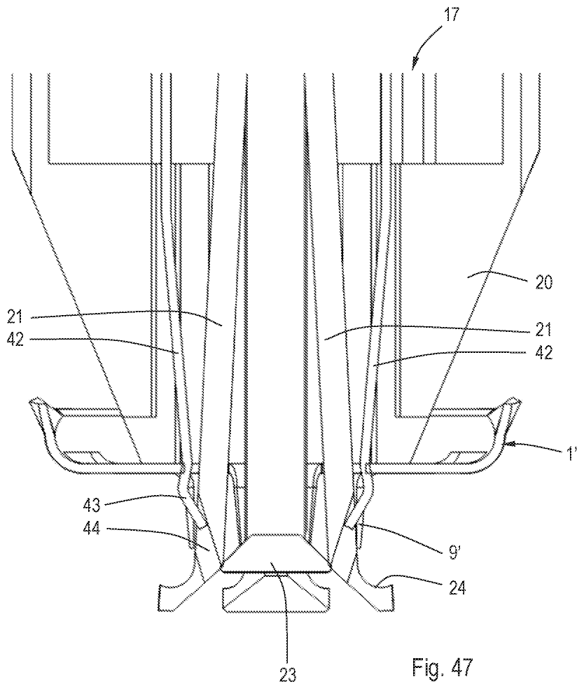

FIGS. 46 and 47 show parts of another alternative embodiment of the applicator 17 which is suitable for mounting the different oval-shaped anastomosis connectors 1, 1′ as shown in FIGS. 37-40 to respective blood vessels. Elements which correspond to elements in the embodiment as shown in FIGS. 43-45 have the same reference numbers. Similar to the latter embodiment the applicator 17 is provided with movable arms 21 including anvils 24 and a spreading element 23 for moving the arms 21 in outward direction. In this case there are two pairs of legs 42 at the respective opposite arms 21. FIG. 47 shows how the legs 42 above their curved free end portions 43 support the anastomosis connector 1′. Under operating conditions, when moving the pusher 20 towards the anvils 24 the free end portions 43 will fall into the respective openings 44 of the arms 21 such that they move inwardly and no longer support the corresponding anastomosis connector 1′, hence releasing it from the applicator 17.

From the foregoing it is clear that the invention provides the opportunity to make a reliable anastomosis between vessels, in particular small blood vessels like coronary artery bypass grafts (CABG). The invention can also be used in other surgical fields, for example vascular surgery including dialysis shunt construction.

The invention is not limited to the embodiments shown in the drawings and described hereinbefore, which may be varied in different manners within the scope of the claims and their technical equivalents. For example, the invention is also applicable for end-to-side or end-to-end anastomosis.

1. An anastomosis connector for being mounted to a flexible vessel, such as a blood vessel, comprising a non-expandable annular body having an inner wall surrounding an anastomotic orifice, an outer wall surrounding the inner wall, a front side and a back side which extend between the inner wall and the outer wall, a plurality of staple pins configured to be bent permanently from a starting position to a final position and located at a distance from each other in circumferential direction of the annular body, wherein the plurality of staple pins project from the annular body and point in a direction away from the back side, wherein a distance between the front side and the back side at the inner wall of the annular body is smaller than a distance between the inner wall and the outer wall.

2. The anastomosis connector according to claim 1, wherein the distance between the front side and the back side at the inner wall of the annular body is smaller than 50%, of the distance between the inner wall and the outer wall.

3. The anastomosis connector according to claim 1, wherein the distance between the inner wall and the outer wall is larger than the a thickness of any of the staple pins as measured in a direction from the inner wall f to the outer wall at a location of the corresponding staple pin.

4. The anastomosis connector according to claim 1, wherein the staple pins are configured to be bent in outward direction of the annular body.

5. The anastomosis connector according to claim 1, wherein the staple pins are located at the inner wall of the annular body.

6. The anastomosis connector according to claim 5, wherein the inner wall between each pair of neighboring staple pins in circumferential direction of the annular body extends substantially linearly.

7. The anastomosis connector according to claim 5, wherein the inner wall between each pair of neighboring staple pins in circumferential direction of the annular body has a recess and wherein the back side of the annular body is provided with a layer of expanded PTFE which also covers the recesses.

8. The anastomosis connector according to claim 5, wherein the inner wall of the annular body is provided with pairs of notches, wherein the notches of each pair are located at either side of each staple pin.

9. The anastomosis connector according to claim 1, wherein the annular body is plate-shaped.

10. The anastomosis connector according to claim 1, wherein at least the front side has a substantially flat surface.

11. The anastomosis connector according to claim 1, wherein the staple pins are substantially linear in their starting position.

12. The anastomosis connector according to claim 1, wherein the back side has a depressed edge portion along the inner wall.

13. The anastomosis connector according to claim 12, wherein the depressed edge portion is surrounded by a layer of material that is different from the material of a remainder of the annular body, for example expanded PTFE.

14. TheAe anastomosis connector according to claim 1, wherein at least a part of the back side of the annular body diverges between the inner wall and the outer wall in a direction from the back side to the front side.

15. The anastomosis connector according to claim 1, wherein the annular body is provided with a reinforcement portion.

16. The anastomosis connector according to claim 15, wherein the reinforcement portion comprises a deformed section of the annular body.

17. The anastomosis connector according to claim 16, wherein the deformed section comprises a circular reinforcement ridge surrounding the anastomotic orifice.

18. The anastomosis connector according to claim 17, wherein the reinforcement ridge forms a protrusion at the back side and a depression at the front side of the annular body.

19. The anastomosis connector according to claim 1, wherein at least one of the inner wall and the back side is treated by a roughening treatment, such as sandblasting and/or acid treatment, wherein surface roughness of the at least one of the inner wall and the back side may be larger than that of the front side.

20. A group of parts, comprising two anastomosis connectors as defined in claim 1 and a fixing member for attaching the annular bodies to each other such that their respective anastomotic orifices are aligned, wherein the front sides of the annular bodies of the respective anastomosis connectors snugly fit to each other.

21. The group of parts according to claim 20, wherein the front sides of the annular bodies of the respective anastomosis connectors snugly fit to each other at different mutual angles about centerlines of their anastomotic orifices.

22. The group of parts according to claim 20, wherein the fixing memberis adapted to attach the annular bodiesreleasably to each other.

23. The group of parts according to claim 20, wherein the anastomosis connectors are substantially identical.

24. The group of parts according to claim 20, wherein the fixing member is provided with a first coupling element at one of the anastomosis connectors which is insertable in a cooperating first coupling member at the other one of the anastomosis connectors when the annular bodies are angled with respect to each other, wherein the first coupling element and the first coupling member are adapted such they form a pivot including a pivot axis at the first coupling element and the first coupling member when the first coupling element is received by the first coupling member, and with a second coupling element at said one of the anastomosis connectors which is attachable to a cooperating second coupling member at said other one of the anastomosis connectors, wherein the first coupling element and the second coupling element are located at a distance from each other.

25. The group of parts according to claim 24, wherein the first coupling member has a hook-shaped cross-section or comprises a hole.

26. The group of parts according to claim 24, wherein the second coupling element and the second coupling member are attachable to each other through a snapping operation upon rotating the mutually angled annular bodies to each other about the pivot axis.

27. The group of parts according to claim 24, wherein the first coupling element and the first coupling member are located at the outer wall of the respective annular bodies.

28. The group of parts according to claim 24, wherein the second coupling element and the second coupling member are located at the outer walls of the respective annular bodies.

29. The group of parts according to claim 20, wherein the fixing member is a separate part.

30. An applicator for mounting an anastomosis connector according to claim 1 to a flexible vessel at an opening thereof, comprising an elongate element and a pusher which are coupled to each other and movable with respect to each other in a direction along a longitudinal axis of the elongate element, wherein the elongate element has a distal end portion to be put through the anastomotic orifice of the anastomosis connector and to be inserted into the opening of the flexible vessel, wherein the distal end portion is provided with a plurality of anvils protruding in outward direction of the elongate element with respect to a longitudinal axis thereof, which anvils have respective upper sides that are directed to the pusher so as to bend the staple pins under operating conditions when the anastomosis connector is located between the pusher and the anvils and the pusher and the anvils are moved to each other.

31. The applicator according to claim 30, wherein the anvils are movable in a transverse direction with respect to the longitudinal axis of the elongate element between retracted positions and extended positions.

32. The applicator according to claim 30, wherein the pusher is provided with a holding device, such as a magnet, for temporarily holding an anastomosis connector.

33. The applicator according to claim 30, wherein the pusher surrounds the elongate element.

34. The applicator according to claim 30, wherein the upper sides of the anvils are concave.

35. The applicator according to claim 30, wherein the applicator is provided with an aligning device for aligning the staple pins of an anastomosis connector with the anvils-.

36. The applicator according to claim 30, 35, wherein the applicator is provided with a vessel supporting member for supporting the vessel at a rim of an opening where the anastomosis connector is to be mounted, wherein the vessel supporting member is located at the distal end portion of the elongate element between the pusher and the anvils.

37. The applicatoraccording to claim 36, wherein the vessel supporting member is expandable in a transverse direction with respect to the longitudinal axis of the elongate element between a retracted position and an expanded position.

38. The applicator according to claim 31, wherein the anvils extend beyond the vessel supporting membe as seen in outward direction of the elongate element when the anvils are in their extended positions and the vessel supporting member is in its expanded position.

39. The applicator according to claim 38, wherein the elongate element comprises a shank and a plurality of arms which extend in longitudinal direction of the shank from an end of the shank to free ends of the arms which arms are movable with respect to the shank in a transverse direction with respect to a longitudinal axis of the shank between retracted positions and extended positions, wherein the distal end portion of the elongate element is formed by distal end portions of the arms, and wherein the applicator is provided with a spreading element which cooperates with the arms and is movable with respect to the arms along the longitudinal axis of the shank, wherein the arms are arranged in circumferential direction about the spreading element and inner sides of the arms and an outer side of the spreading element are adapted such that the arms are moved between their retracted positions and their extended positions upon moving the spreading element with respect to the arms, wherein the vessel supporting member is formed by outer surfaces of the respective arms at the distal end portions thereof and the anvils are also provided at the distal end portions.

40. The applicator according to claim 39, wherein the outer surfaces of the arms are provided with respective recesses extending in longitudinal directions thereof, for receiving free ends of passing staple pins of the anastomosis connector when the pusher and the anvils are moved to each other.

41. The applicator according to claim 39, wherein the spreading element is mounted to a stem which extends through the shank such that the stem and the shank are movable with respect to each other in their longitudinal directions.

42. The applicator according to claim 30, wherein the applicator is provided with a holding device for temporarily holding an anastomosis connector to the pusher, wherein the holding device comprises jaws which are movable with respect to the pusher between holding positions for holding an anastomosis connector to the pusher and releasing positions for disconnecting an anastomosis connector from the pusher.

43. The applicator according to claim 42, wherein the applicator has a housing, and the jaws are rotatably mounted to the housing.

44. The applicator according to claim 42, wherein the jaws and the pusher cooperate such that during movement of the pusher towards the anvils the pusher moves the jaws from their holding positions to their releasing positions.

45. The applicator according to claim 43, wherein the jaws are fixed to respective bi-stable springs, which are switched from a first stable shape to a second stable shape during movement of the pusher towards the anvils.

46. The applicator according to claim 39, wherein the applicator is provided with a holding device for temporarily holding an anastomosis connector to the pusher, wherein the holding device comprises at least a leg between one of the arms and the pusher, which leg is movable with respect to the arm and the pusher in longitudinal direction of the shank, wherein the leg has a free end portion and the arm has an opening, wherein the free end portion has a supporting section for supporting an anastomosis connector, wherein the arm, the leg and the pusher are adapted such that during moving the pusher with respect to the arm in its extended position in a direction towards the anvils the pusher moves the leg in the same direction such that the free end portion slides along the arm and falls into the opening at a predetermined relative position of the arm and the leg.

47. (canceled)
